(19) United States
(12) Patent Application Publication
(10) Pub. No.: US 2024/0182435 A1
(43) Pub. Date: Jun. 6, 2024

(54) CHEMICAL SYNTHESIS METHOD OF PRODELPHINIDIN B9 GALLATE

(71) Applicant: ZHEJIANG UNIVERSITY, Hangzhou, Zhejiang (CN)

(72) Inventors: Shiguo CHEN, Hangzhou (CN); Xinlei XU, Hangzhou (CN); Haibo PAN, Hangzhou (CN); Xingqian YE, Hangzhou (CN); Yi WANG, Hangzhou (CN); Huan CHENG, Hangzhou (CN); Jianle CHEN, Hangzhou (CN)

(21) Appl. No.: 17/779,274

(22) PCT Filed: May 21, 2021
(86) PCT No.: PCT/CN2021/095046

§ 371 Date: May 24, 2022

(51) Int. Cl. C07D 311/64

(52) U.S. Cl. CPC C07D 311/64

(57) ABSTRACT

The disclosure aims to provide a chemical synthesis method of a prodelphinidin B9 gallate, in which the prodelphinidin B9 gallate is synthesized by using a proanthocyanidin from Chinese bayberry leaves as a raw material, and using epigallocatechin gallate (EGCG) as a nucleophilic reagent to attack C4 sites of subunits EGCG and epigallocatechin (EGC) of the proanthocyanidin from Chinese bayberry leaves in the presence of hydrochloric acid as a catalyst. Compared with prodelphinidin B9 gallates extracted and separated from materials such as tea leaves, and Chinese bayberry leaves, the prodelphinidin B9 gallate prepared by the method provided in the present disclosure has higher purity and yield, and can be directly used as a nutrient enhancer and a natural antioxidant in the field of food.

### TECHNICAL FIELD

The present disclosure relates to the field of plant functional components, and in particular to a method for preparing a prodelphinidin B9 gallate.

### BACKGROUND ART

Proanthocyanidins, a kind of polymeric polyphenols with flavan-3-ols as subunits, widely exist in plants, and are the second most abundant dietary polyphenols after lignin. Proanthocyanidins are mainly composed of subunits (epi)catechin, (epi)afzelechin, (epi)gallocatechin((E)GC) and gallates thereof. According to a large number of researches, it has been shown that proanthocyanidins have various of beneficial biological activities such as oxidation resistance, blood glucose reduction and weight loss. These functions are closely related to the structure of proanthocyanidins, especially types of the subunits and polymerization degrees. Firstly, bioavailability of proanthocyanidins is determined by the polymerization degrees. With the increase of the polymerization degree, the bioavailability decreases. Proanthocyanidins with a polymerization degree greater than 4 are basically not absorbed. A density of a phenolic hydroxyl group in a subunit of the proanthocyanidins is closely related to the biological activity thereof. It has been shown that compared with proanthocyanidins without galloyl groups, proanthocyanidins with galloyl groups in the subunits exhibit stronger biological activity due to a higher density of the phenolic hydroxyl group. Prodelphinidins with (E)GC and gallate thereof ((E)GCG) as subunits have strong activity.

Proanthocyanidins that are widely studied nowadays include (epi)catechin, (epi)afzelechin and gallates thereof as subunits. Due to abundant sources of the proanthocyanidins, study on structure-function relationships of dimers thereof is relatively clear. While study on prodelphinidins is carried out based on mixtures of proanthocyanidins. The proanthocyanidins derived from Chinese bayberry leaves have typical prodelphinidin structures, include EGC and EGCG as main subunits, and have extremely strong in-vitro antioxidant activity. However, due to their high polymerization degree (being mostly between 9.5 and 26.7), the proanthocyanidins derived from Chinese bayberry leaves have an extremely low bioavailability, a low in-vivo activity, and an unclear action mechanisms. Prodelphinidins with a low polymerization degree are extremely low in amount and difficult for separation purification, resulting in a limited study on the structure-function relationships thereof.

### SUMMARY

An object of the present disclosure is to provide a chemical synthesis method of a prodelphinidin B9 gallate. In the present disclosure, with a proanthocyanidin from Chinese bayberry leaves as a raw material, and EGCG as a nucleophilic substrate, two dimeric prodelphinidin gallates (a prodelphinidin B-3′-gallate and a prodelphinidin B-3,3′-digallate) are synthesized in the presence of an acid as a catalyst, which enables a component content to be increased, and purification difficulty to be further reduced. The obtained dimeric prodelphinidin gallates are B-type proanthocyanidin dimers with EGCG as a subunit, which not only have an improved bioavailability of the prodelphinidin-type proanthocyanidin, but also provide high-purity materials for a study on the structure-function relationship of the prodelphinidin-type proanthocyanidin.

The present disclosure provides the following technical solutions.

Provided is a chemical synthesis method of a prodelphinidin B9 gallate, specifically including:
separately preparing a hydrochloric acid-containing solution of a proanthocyanidin from Chinese bayberry leaves with a concentration of 10 mg/mL to 400 mg/mL and a hydrochloric acid-containing solution of EGCG with a concentration of 10 mg/mL to 400 mg/mL, where both the hydrochloric acid-containing solutions have a hydrochloric acid concentration of 0.1 mol/L to 1.0 mol/L; and
mixing the hydrochloric acid-containing solution of the proanthocyanidin from Chinese bayberry leaves and the hydrochloric acid-containing solution of EGCG with the same hydrochloric acid concentration at a volume ratio of 1:2 to 2:1 to obtain a mixed solution, subjecting the mixed solution to a reaction at 20-60° C. for 40 min to obtain a reacted solution, and subjecting the reacted solution to a drying and a separation purification to obtain the prodelphinidin B9 gallate, wherein the prodelphinidin B9 gallate includes prodelphinidin B-3′-gallate and prodelphinidin B-3,3′-digallate.

The prodelphinidin B-3′-gallate and the prodelphinidin B-3,3′-digallate each have structural formula as follows:

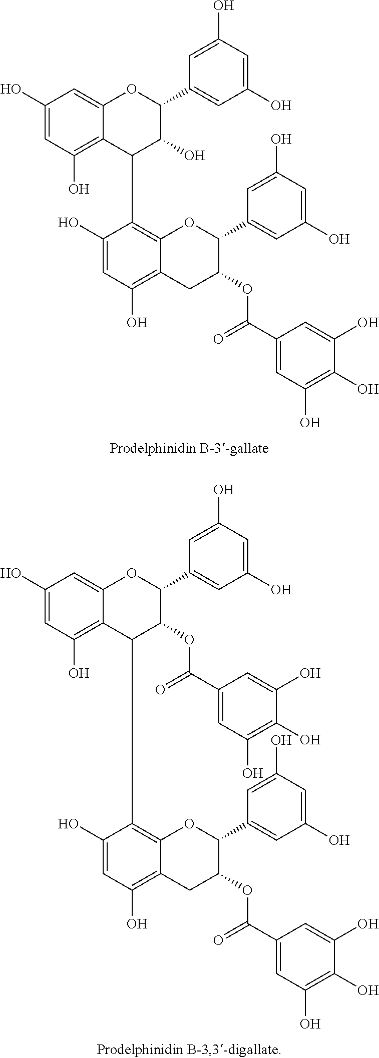 [SMILES: O=C(O[C@@H]1Cc2c(O)cc(O)c(C3c4c(O)cc(O)cc4O[C@H](c4cc(O)cc(O)c4)[C@@H]3O)c2O[C@@H]1c1cc(O)cc(O)c1)c1cc(O)c(O)c(O)c1 ; O=C(O[C@@H]1C(c2c(O)cc(O)c3c2O[C@H](c2cc(O)cc(O)c2)[C@H](OC(=O)c2cc(O)c(O)c(O)c2)C3)c2c(O)cc(O)cc2O[C@@H]1c1cc(O)cc(O)c1)c1cc(O)c(O)c(O)c1]

The proanthocyanidin from Chinese bayberry leaves is separated and purified by a method described in a patent entitled “Method for preparing proanthocyanidin from Chinese bayberry leaves by separation” (CN201310181254.3). The obtained proanthocyanidin from Chinese bayberry leaves therein had a purity of 86.4%. EGCG is a commercially available reagent with high performance liquid chromatography (HPLC) grade and a purity greater than or equal to 98%.

In some embodiments, the drying specifically includes the following steps: subjecting the reacted solution obtained after reacting for 40 min to a rotary evaporation at 40° C. to remove methanol and a small amount of water, so as to obtain a dry powder of a crude reaction product.

In some embodiments, the separation purification is conducted on the obtained crude reaction product by a Shimadzu LC-20 semi-preparative liquid chromatography to obtain the prodelphinidin B9 gallate.

The separation purification includes the following steps:
using an S series X5H preparation column (10 mm*250 mm, 5 μm), acetonitrile containing 0.5 vol % acetic acid as a mobile phase A, and ultrapure water as a mobile phase B;
dissolving the crude reaction product in a mixture of the mobile phase A and the mobile phase B with a volume ratio of 88:12 to prepare a solution with a concentration of the crude reaction product of 20 mg/mL as a sample;
injecting 2 mL of the sample into the S series X5H preparation column, and purifying the sample by elution using the mobile phases under conditions of
   a flow rate of 5 mL/min, and
   an elution gradient: 12 vol % to 27 vol % of the mobile phase B, from 0 min to 10 min; 60 vol % of the mobile phase B, from 10 min to 20 min; and 12 vol % of the mobile phase B, from 20 min to 26 min; and
setting a detection wavelength to be 280 nm, collecting an effluent at a retention time from 5.5 min to 6.0 min according to a liquid phase spectrum, subjecting the effluent to a rotary evaporation at 40° C. to remove acetonitrile, and subjecting the effluent after the rotary evaporation to a freeze-drying to obtain a purified prodelphinidin B9 gallate.

In some embodiments, the separation purification further includes a second purification, which includes the following steps:
using a Shim-pack GIST C18 column (20 mm*250 mm, 5 μm), ultrapure water as a mobile phase A and 98 vol % acetonitrile (containing 0.05 vol % phosphoric acid) as a mobile phase B;
dissolving the purified prodelphinidin B9 gallate in a mixture of the mobile phase A and the mobile phase B with a volume ratio of 80:20 to prepare a solution with a concentration of the purified prodelphinidin B9 gallate of 50 mg/mL as a sample;
injecting 2 mL of the sample into the Shim-pack GIST C18 column, and purifying the sample by elution using the mobile phases under conditions of
   a flow rate of 15 mL/min, and
   an elution gradient: 20 vol % to 40 vol % of the mobile phase B, from 0 min to 5 min; and 40 vol % of the mobile phase B, from 5 min to 15 min; and
setting a detection wavelength to be 280 nm, separately collecting effluents at different peak times according to a liquid phase spectrum, subjecting the effluents to a rotary evaporation at 40° C. to remove acetonitrile, and subjecting the effluents after the rotary evaporation to a freeze-drying to obtain prodelphinidin B-3′-gallate and prodelphinidin B-3,3′-digallate.

In the present disclosure, a prodelphinidin B9 gallate is synthesized by using a proanthocyanidin from Chinese bayberry leaves as a raw material, and using EGCG as a nucleophilic reagent to attack C4 sites of the subunits EGCG and EGC of the proanthocyanidin from Chinese bayberry leaves in the presence of hydrochloric acid as a catalyst. Compared with prodelphinidin B9 gallates extracted and separated from materials such as tea leaves and Chinese bayberry leaves, the prodelphinidin B9 gallate prepared by the method provided in the present disclosure has a relatively high purity and yield, and could be directly used as a nutrient enhancer and a natural antioxidant in the field of food.

### BRIEF DESCRIPTION OF THE DRAWINGS

FIG. 1 shows a combined HPLC analysis spectrum of the prodelphinidin B9 gallate in Example 1.

FIG. 2 shows a combined HPLC analysis spectrum of the prodelphinidin B9 gallate in Example 2.

FIG. 3 shows a combined HPLC analysis spectrum of the prodelphinidin B9 gallate in Example 3.

FIG. 4 shows a combined HPLC analysis spectrum of the prodelphinidin B9 gallate in Example 4.

FIG. 5 shows a combined HPLC analysis spectrum of the prodelphinidin B9 gallate in Example 5.

FIG. 6 shows a combined HPLC analysis spectrum of the prodelphinidin B9 gallate in Example 6.

FIG. 7 shows an HPLC analysis spectrum of the prodelphinidin B-3′-gallate with a purity of 93.2% prepared in Example 1.

FIG. 8 shows an HPLC analysis spectrum of the prodelphinidin B-3,3′-digallate with a purity of 96.6% prepared in Example 1.

FIG. 9 shows a mass spectrum of the prodelphinidin B-3′-gallate with a molecular weight of 762 prepared in Example 1.

FIG. 10 shows a mass spectrum of the prodelphinidin B-3,3′-digallate with a molecular weight of 914 prepared in Example 1.

FIG. 11 shows a carbon-13 nuclear magnetic resonance (¹³C NMR) spectrum of the prodelphinidin B-3′-gallate prepared in Example 1.

FIG. 12 shows a ¹³C NMR spectrum of the prodelphinidin B-3,3′-digallate prepared in Example 1.

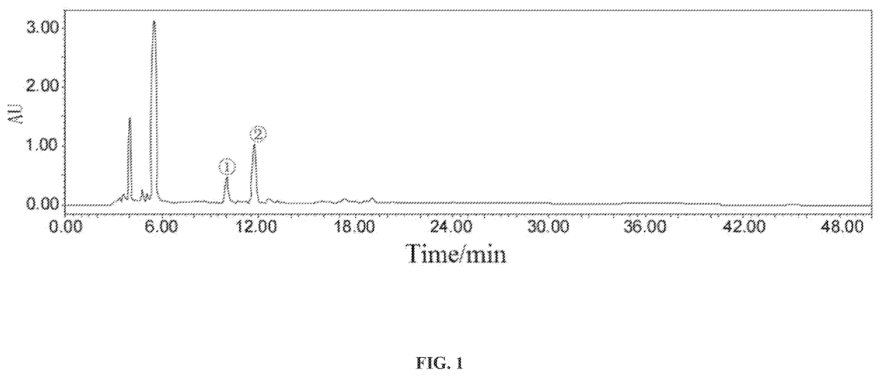

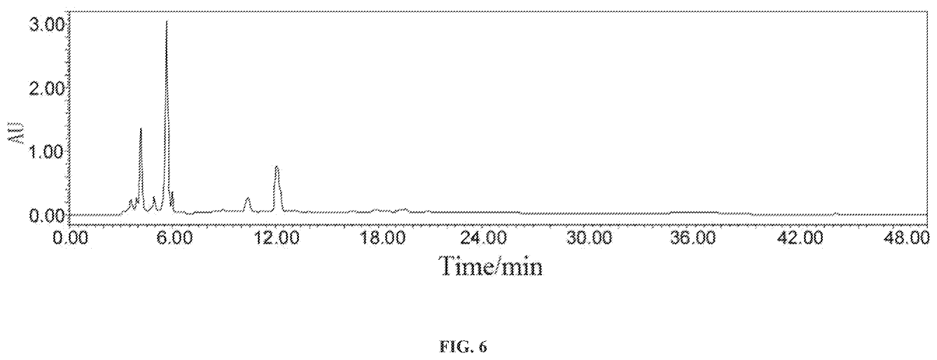

In FIG. 1 to FIG. 6, the prodelphinidin B-3′-gallate (1) and the prodelphinidin B-3,3′-digallate (2) have a peak time of 9.9 min and 11.7 min, respectively.

### DETAILED DESCRIPTION OF THE EMBODIMENTS

In order to make the objects and technical solutions of the present disclosure clearer, specific embodiments of the present disclosure are further described in detail below, but are not intended to limit the present disclosure. In various examples, the differences between them lie in reaction conditions. and drying and separation purification (with two purifications) are specified as follows:

Drying: the reacted solution obtained after reacting for 40 min is subjected to a rotary evaporation to remove methanol and a small amount of water, so as to obtain a dry powder of a crude reaction product.

Separation Purification:
a first purification is conducted as follows: an S series X5H preparation column (10 mm* 250 mm, 5 μm) is used, acetonitrile containing 0.5 vol % acetic acid is used as a mobile phase A, and ultrapure water is used as a mobile phase B; the crude reaction product is dissolved in a mixture of the mobile phase A and the mobile phase B with a volume ratio of 88:12 to prepare a solution with a concentration of the crude reaction product of 20 mg/mL as a sample; 2 mL of the sample is injected into the S series X5H preparation column, and purified by elution using the mobile phases at a flow rate of 5 mL/min, with an elution gradient being 12 vol % to 27 vol % of the mobile phase B, from 0 min to 10 min, 60 vol % of the mobile phase B, from 10 min to 20 min, and 12 vol % of the mobile phase B, from 20 min to 26 min; and a detection wavelength is set to be 280 nm, an effluent is collected at a retention time from 5.5 min to 6.0 min according to a liquid phase spectrum, the effluent is subjected to a rotary evaporation at 40° C. to remove acetonitrile, and then the effluent after the rotary evaporation is subjected to a freeze-drying to obtain a purified prodelphinidin B9 gallate; and
a second purification is conducted as follows: a Shim-pack GIST C18 column (20 mm*250 mm, 5 μm) is used, ultrapure water is used as a mobile phase A, and 98 vol % acetonitrile (containing 0.05 vol % phosphoric acid) is used as a mobile phase B; the purified prodelphinidin B9 gallate is dissolved in a mixture of the mobile phase A and the mobile phase B with a volume ratio of 80:20 to prepare a solution with a concentration of the purified prodelphinidin B9 gallate of 50 mg/mL as a sample; 2 mL of the sample is injected into the Shim-pack GIST C18 column, and then purified by elution using the mobile phases at a flow rate of 15 mL/min, with an elution gradient being 20 vol % to 40 vol % of the mobile phase B, from 0 min to 5 min, and 40 vol % of the mobile phase B, from 5 min to 15 min; and a detection wavelength is set to be 280 nm, effluents are separately collected at different peak times according to a liquid phase spectrum, the effluents are respectively subjected to a rotary evaporation at 40° C. to remove acetonitrile, and the effluents after the rotary evaporation are then subjected to a freeze-drying to obtain prodelphinidin B-3′-gallate and prodelphinidin B-3,3′-digallate.

In addition, a method for detecting the prepared prodelphinidin B9 gallate is conducted as follows:
the prodelphinidin B9 gallate is analyzed by HPLC under the following specific conditions: Waters 2695 is used as the HPLC, a Waters 2489 ultraviolet-visible light detector is used as a detector, with a detection wavelength being 280 nm, and a Chromatographic column used is Luna hilic (4.6 mm*250 mm, 5 Phenomemex Inc.); acetonitrile (containing 0.5 vol % acetic acid) is used as a mobile phase A, and ultrapure water is used as a mobile phase B; an elution gradient includes 12 vol % to 27 vol % of the mobile phase B, from 0 min to 30 min, 27 vol % to 80 vol % of the mobile phase B, from 30 min to 45 min, 80 vol % to 12 vol % of the mobile phase B, from 45 min to 46 min, and 12 vol % of the mobile phase B, from 46 min to 55 min; a flow rate is 0.8 mL/min; a sample injection volumn is 10 μL; and a column temperature is 30° C.

A method for determining the purity and structure of the prepared prodelphinidin B9 gallate is conducted as follows:
a liquid chromatography-mass spectrometry analysis: a rapid liquid chromatography-triple quadrupole mass spectrometer Agilent 1290/6460 Triple Quad is used for analysis under the same chromatographic conditions as those described above and mass spectrometry conditions that an ESI negative ion mode is used as an ionization mode; a scanning range of a mass-to-charge ratio is 50 m/z to 1,500 m/z; a desolvation temperature is 400° C.; a capillary voltage is 3.5 kV; and a collision energy is 16 eV.
¹³C NMR analysis: an Agilent DD2-600 NMR spectrometer with a superconducting magnet is used for analysis with methanol-D as a solvent.

### Example 1

1 g of a proanthocyanidin from Chinese bayberry leaves and 1 g of EGCG were separately weighed, and dissolved in 50 mL of a 0.1 mol/L HCl methanol solution by stirring to be uniform until they were clear, to obtain a solution of the proanthocyanidin from Chinese bayberry leaves and a solution of EGCG. 50 mL of the solution of the proanthocyanidin from Chinese bayberry leaves and 50 mL of the solution of EGCG were mixed in a conical flask with a cover and heated in a water bath at 40° C. for 40 min to obtain a reacted solution. The reacted solution was dried, subjected to a separation purification and then a detection. As shown in FIG. 1, the prodelphinidin B-3′-gallate had a yield of 8.5%, and the prodelphinidin B-3,3′-digallate had a yield of 20.0%.

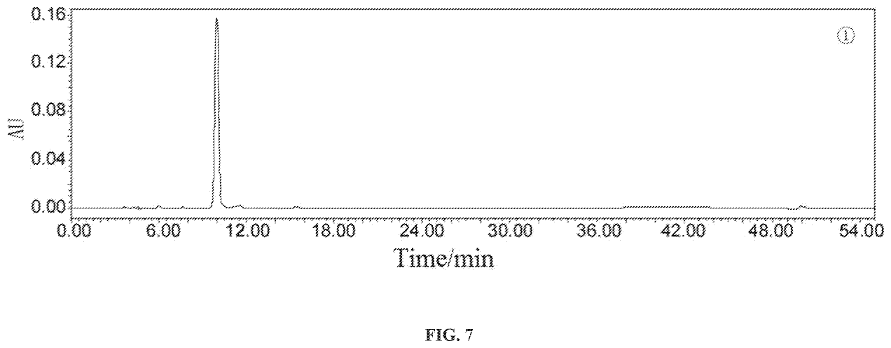

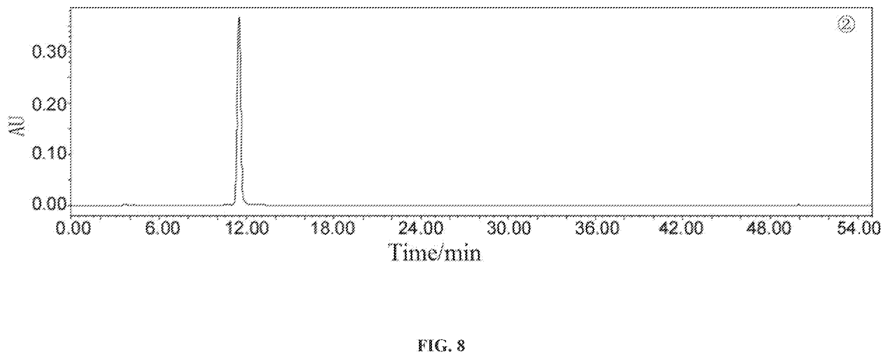

FIG. 7 and FIG. 8 shows HPLC analysis spectras of the prepared prodelphinidin B-3′-gallate and prodelphinidin B-3,3′-digallate, respectively. The prodelphinidin B-3′-gallate had a purity of 93.2%, and the prodelphinidin B-3,3′-digallate had a purity of 96.6%.

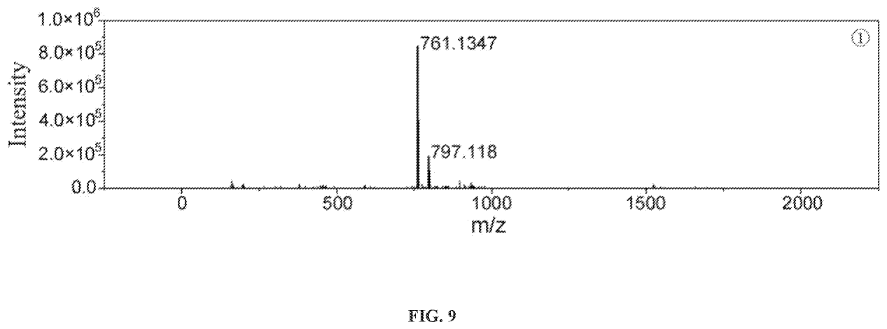

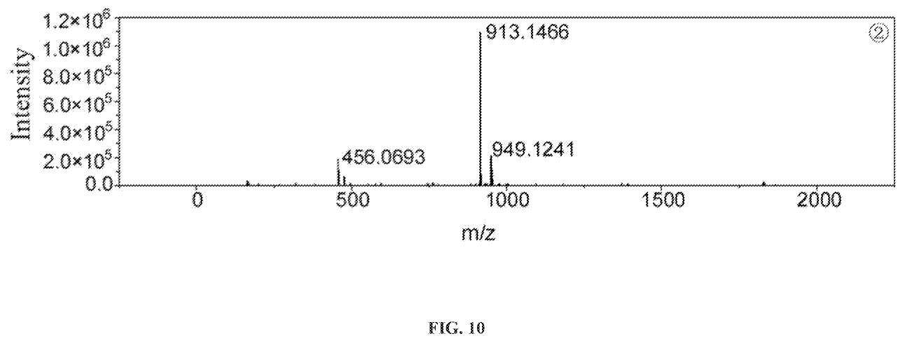

FIG. 9 and FIG. 10 shows mass spectras of the prodelphinidin B-3′-gallate and the prodelphinidin B-3,3′-digallate prepared in the present disclosure, respectively. It can be seen that the prodelphinidin B-3′-gallate had a molecular weight of 762, and the prodelphinidin B-3,3′-digallate had a molecular weight of 914.

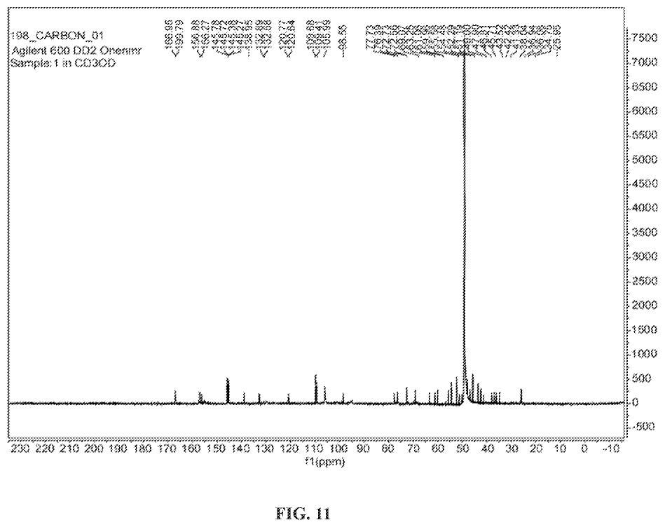

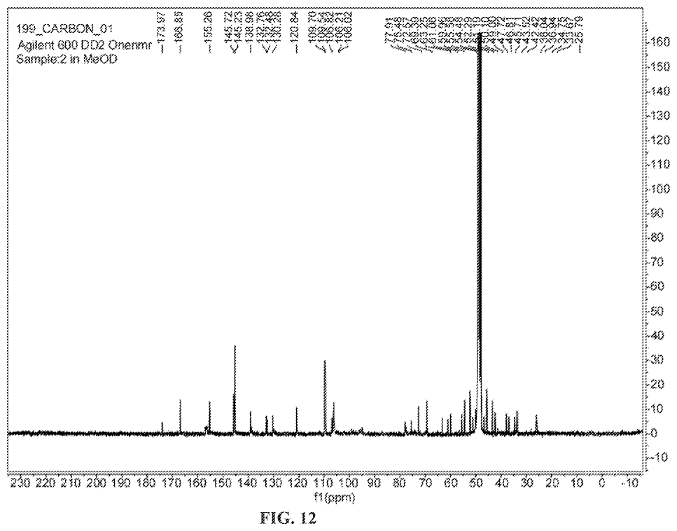

FIG. 11 and FIG. 12 shows 13° C. NMR spectras of the prodelphinidin B-3′-gallate and the prodelphinidin B-3,3′-digallate prepared in the present disclosure, respectively. A carbonyl group signal (166 ppm) and galloyl ring carbon signals (138 ppm and 110 ppm) of gallic acid were characteristics of gallated flavan-3-ols, indicating that a subunit contains EGCG. In addition, an A-type proanthocyanidin usually had a broad peak signal at 102-104 ppm, but the signal was not detected, indicating that there was no A-type bond in the structure. These prove that prodelphinidin B-3′-gallate and prodelphinidin B-3,3′-digallate were obtained.

### Example 2

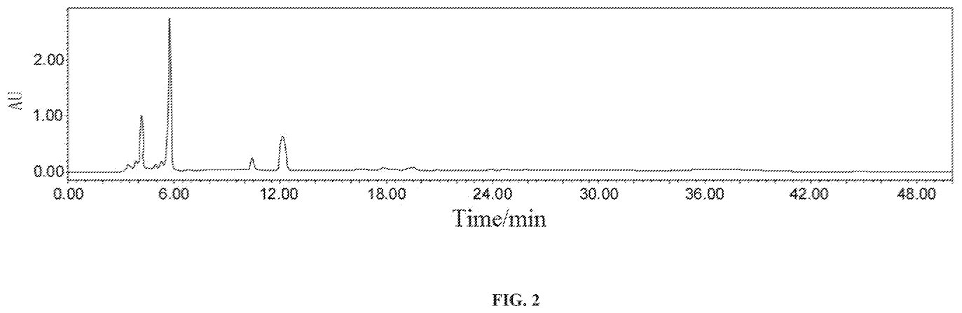

1 g of a proanthocyanidin from Chinese bayberry leaves and 1 g of EGCG were separately weighed, and dissolved in 100 mL of a 0.1 mol/L HCl methanol solution by stirring to be uniform until they were clear, to obtain a solution of the proanthocyanidin from Chinese bayberry leaves and a solution of EGCG. 50 mL of the solution of the proanthocyanidin from Chinese bayberry leaves and 25 mL of the solution of EGCG were mixed in a conical flask with a cover and heated in a water bath at 40° C. for 40 min to obtain a reacted solution. The reacted solution was dried, and subjected to a separation purification and then a detection. As shown in FIG. 2, the prodelphinidin B-3′-gallate had a yield of 6.2%, and the prodelphinidin B-3,3′-digallate had a yield of 11.6%.

### Example 3

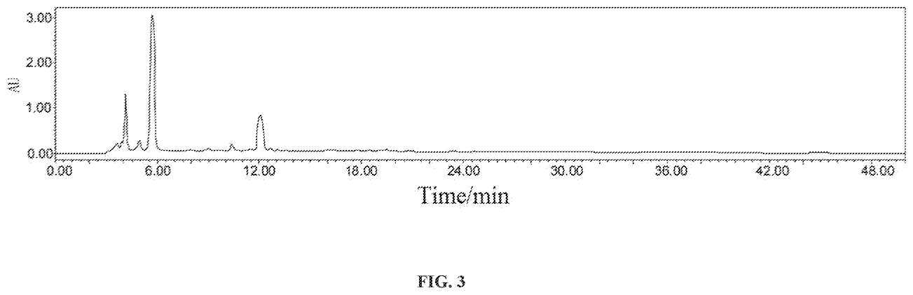

1 g of a proanthocyanidin from Chinese bayberry leaves and 1 g of EGCG were separately weighed, and dissolved in 50 mL of a 0.1 mol/L HCl methanol solution by stirring to be uniform until they were clear, to obtain a solution of the proanthocyanidin from Chinese bayberry leaves and a solution of EGCG. 50 mL of the solution of the proanthocyanidin from Chinese bayberry leaves and 50 mL of the solution of EGCG were mixed in a conical flask with a cover and heated in a water bath at 60° C. for 40 min to obtain a reacted solution. The reacted solution was dried, and subjected to a separation purification and then a detection. As shown in FIG. 3, the prodelphinidin B-3′-gallate had a yield of 4.0%, and the prodelphinidin B-3,3′-digallate had a yield of 18.0%.

### Example 4

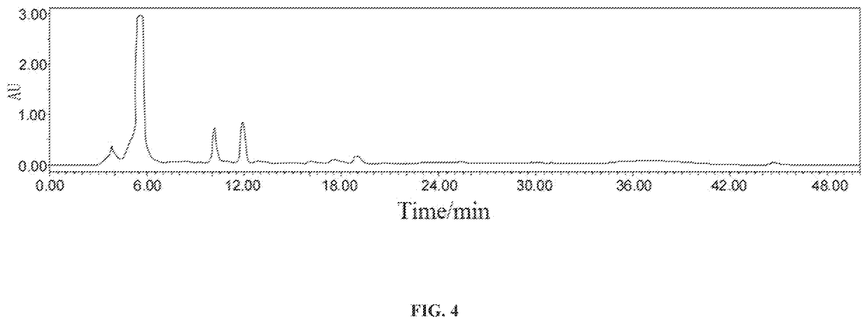

1 g of a proanthocyanidin from Chinese bayberry leaves and 1 g of EGCG were separately weighed, and dissolved in 5.0 mL of a 0.1 mol/L HCl methanol solution by stirring to be uniform until they were clear, to obtain a solution of the proanthocyanidin from Chinese bayberry leaves and a solution of EGCG. 2.5 mL of the solution of the proanthocyanidin from Chinese bayberry leaves and 5.0 mL of the solution of EGCG were mixed in a conical flask with a cover and heated in a water bath at 20° C. for 40 min to obtain a reacted solution. The reacted solution was dried, and subjected to a separation purification and then a detection. As shown in FIG. 4, the prodelphinidin B-3′-gallate had a yield of 16.6%, and the prodelphinidin B-3,3′-digallate had a yield of 19.5%.

### Example 5

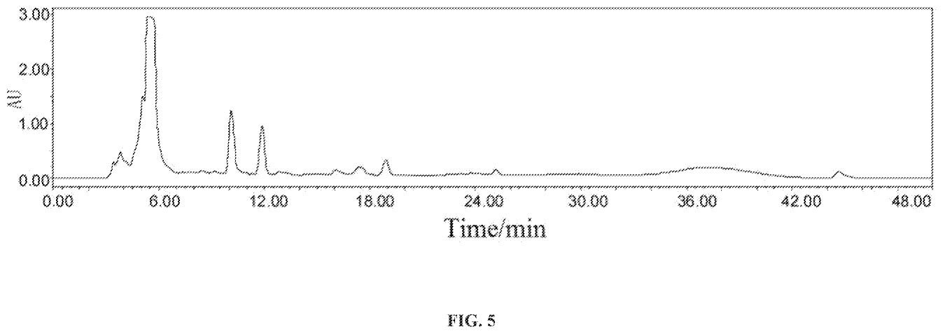

2 g of a proanthocyanidin from Chinese bayberry leaves and 2 g of EGCG were separately weighed and dissolved in 5.0 mL of a 0.1 mol/L HCl methanol solution by stirring to be uniform until they were clear, to obtain a solution of the proanthocyanidin from Chinese bayberry leaves and a solution of EGCG. 2.5 mL of the solution of the proanthocyanidin from Chinese bayberry leaves and 5.0 mL of the solution of EGCG were mixed in a conical flask with a cover and heated in a water bath at 40° C. for 40 min to obtain a reacted solution. The reacted solution was dried, and subjected to a separation purification and then a detection. As shown in FIG. 5, the prodelphinidin B-3′-gallate had a yield of 27.9%, and the prodelphinidin B-3,3′-digallate had a yield of 21.2%.

### Example 6

1 g of a proanthocyanidin from Chinese bayberry leaves and 1 g of EGCG were separately weighed, and dissolved in 50.0 mL of a 1 mol/L HCl methanol solution by stirring to be uniform until they were clear, to obtain a solution of the proanthocyanidin from Chinese bayberry leaves and a solution of EGCG. 50 mL of the solution of the proanthocyanidin from Chinese bayberry leaves and 50 mL of the solution of EGCG were mixed in a conical flask with a cover and heated in a water bath at 40° C. for 40 min to obtain a reacted solution. The reacted solution was dried, and subjected to a separation purification and then a detection. As shown in FIG. 4, the prodelphinidin B-3′-gallate had a yield of 6.8%, and the prodelphinidin B-3,3′-digallate had a yield of 18.0%.

The above descriptions are only preferred embodiments of the present disclosure, and are not intended to limit the scope of patent protection of the present disclosure. Therefore, any equivalent structural changes made by using the contents of the description of the present disclosure shall fall within the protection scope of the appended claims of the present disclosure.

What is claimed is:

1. A chemical synthesis method of a prodelphinidin B9 gallate, specifically comprising:
separately preparing a hydrochloric acid-containing solution of a proanthocyanidin from Chinese bayberry leaves with a concentration of 10 mg/mL to 400 mg/mL and a hydrochloric acid-containing solution of epigallocatechin gallate (EGCG) with a concentration of 10 mg/mL to 400 mg/mL, wherein both the hydrochloric acid-containing solutions have a hydrochloric acid concentration of 0.1 mol/L to 1.0 mol/L; and
mixing the hydrochloric acid-containing solution of the proanthocyanidin from Chinese bayberry leaves and the hydrochloric acid-containing solution of EGCG with the same hydrochloric acid concentration with a volume ratio of 1:2 to 2:1 to obtain a mixed solution, subjecting the mixed solution to a reaction at 20-60° C. for 40 min to obtain a reacted solution, and subjecting the reacted solution to a drying and a separation purification to obtain the prodelphinidin B9 gallate, wherein the prodelphinidin B9 gallate comprises prodelphinidin B-3′-gallate and prodelphinidin B-3,3′-digallate.

2. The chemical synthesis method of claim 1, wherein a solvent used for separately preparing the hydrochloric acid-containing solution of the proanthocyanidin from Chinese bayberry leaves with a concentration of 10 mg/mL to 400 mg/mL and the hydrochloric acid-containing solution of EGCG with a concentration of 10 mg/mL to 400 mg/mL comprises one selected from the group consisting of methanol and ethanol.

3. The chemical synthesis method of claim 1, wherein the proanthocyanidin from Chinese bayberry leaves has a purity of 86.4%, and the EGCG has a purity greater than or equal to 98%.

4. The chemical synthesis method of claim 1, wherein the drying specifically comprises:
subjecting the reacted solution obtained after reacting for 40 min to a rotary evaporation at 40° C. to remove methanol and a small amount of water, so as to obtain a dry powder of a crude reaction product.

5. The chemical synthesis method of claim 1, wherein the separation purification specifically comprises:
using an S series X5H preparation column, acetonitrile containing 0.5 vol % acetic acid as a mobile phase A and ultrapure water as a mobile phase B;
dissolving a crude reaction product obtained after the drying in a mixture of the mobile phase A and the mobile phase B with a volume ratio of 88:12 to prepare a solution with a concentration of the crude reaction product of 20 mg/mL as a sample;
injecting 2 mL of the sample into the S series X5H preparation column, and purifying the sample by elution using the mobile phases under conditions of
a flow rate of 5 mL/min; and
an elution gradient: 12 vol % to 27 vol % of the mobile phase B, from 0 min to 10 min; 60 vol % of the mobile phase B, from 10 min to 20 min; and 12 vol % of the mobile phase B, from 20 min to 26 min; and
setting a detection wavelength to be 280 nm, collecting an effluent at a retention time from 5.5 min to 6.0 min according to a liquid phase spectrum, subjecting the effluent to a rotary evaporation at 40° C. to remove acetonitrile, and subjecting the effluent after the rotary evaporation to a freeze-drying to obtain a purified prodelphinidin B9 gallate.

6. The chemical synthesis method of claim 5, wherein the separation purification further comprises a second purification, which comprises the following steps:
using a Shim-pack GIST C18 column, ultrapure water as a mobile phase A and 98 vol % acetonitrile containing 0.05 vol % phosphoric acid as a mobile phase B;
dissolving the purified prodelphinidin B9 gallate in a mixture of the mobile phase A and the mobile phase B with a volume ratio of 80:20 to prepare a solution with a concentration of the purified prodelphinidin B9 gallate of 50 mg/mL as a sample;
injecting 2 mL of the sample into the Shim-pack GIST C18 column, and purifying the sample by elution using the mobile phases under conditions of:
a flow rate of 15 mL/min; and
an elution gradient: 20 vol % to 40 vol % of mobile phase B, from 0 min to 5 min; and 40 vol % of the mobile phase B, from 5 min to 15 min; and
setting a detection wavelength to be 280 nm, separately collecting effluents at different peak times according to a liquid phase spectrum, subjecting the effluents to a rotary evaporation at 40° C. to remove acetonitrile, and subjecting the effluents after the rotary evaporation to a freeze-drying to obtain prodelphinidin B-3′-gallate and prodelphinidin B-3,3′-digallate.
